(19) United States
(12) Patent Application Publication
(10) Pub. No.: US 2002/0034776 A1
(43) Pub. Date: Mar. 21, 2002

(54) ALTERATION OF THE SUBSTRATE SPECIFICITY OF ENZYMES

(76) Inventors: UWE BORNSCHEUER, STUTTGART (DE); HARTMUT HERMANN MEYER, HANNOVER (DE); JOSEF ALTENBUCHNER, NUFRINGEN (DE)

Correspondence Address: HERBERT B KEIL, KEIL & WEINKAUF, 1101 CONNECTICUT AVE NW, WASHINGTON, DC, 20036

(21) Appl. No.: 09/161,680

(22) Filed: Sep. 28, 1998

(30) Foreign Application Priority Data
Oct. 2, 1997 (DE) 19743683.8

(51) Int. Cl. C12P 21/06

(52) U.S. Cl. 435/69.1

(57) ABSTRACT

A method for altering the substrate specificity of enzymes is described.

The invention relates to a method for altering the substrate specificity of enzymes.

The synthetic chemist is frequently confronted by the problem in practice that he is unable to follow synthetic routes which are easy and simple to carry out to prepare a chemical compound because these routes would result in compounds in which, for example, the elimination of a required protective group is no longer possible because, otherwise, the synthetized molecule would be destroyed. To solve this problem he frequently has to follow a more complicated and time-consuming synthetic route.

Enzymes cleave chemical bonds under mild conditions. They can therefore sometimes be used to solve synthetic problems of this type, ie. a protective group can, for example, be eliminated enzymatically under mild conditions without destroying other bonds and thus the molecule. Enzymes thus make it possible to obtain the required substances easily and quickly in the laboratory. However, the enzymatic activity and/or stability is often inadequate for industrial use of the enzymes, so that although the chemical syntheses require a larger number of synthesis stages, nevertheless they are less costly and are therefore implemented industrially.

This is why a large number of studies have been carried out on improving the enzymatic activity and/or stability. Various routes have been followed for this.

It is possible by site directed mutagenesis to achieve very specific improvements in the stability and/or enzymatic activity of enzymes. The disadvantage of site directed mutagenesis of enzymes is that this method requires the availability of a large amount of knowledge about the structure and function of the enzymes from X-ray structural analyses, from modeling, and from comparisons with other enzymes of the same or similar specificity. In addition, the sequence of the structural genes must be known in order for it in fact to be possible to improve the enzymes in a targeted manner. This information is, as a rule, unavailable or not yet or only partly available, so that, in these cases, this method usually does not have the desired result because it is unclear which regions of the enzyme require alteration. If only little information about the enzymatic activity is available, preference is to be given to methods which involve a random strategy to improve the enzyme, although these methods should nevertheless be as targeted as possible.

Thus, Spee et al. (Nucleic Acids Research, Vol. 21, No. 3, 1993: 777-778) describe a PCR method using dITP for the random mutagenesis of the nisZ gene of Lactococcus lactis.

The method described by Spee et al. was further improved by Rellos et al. (Protein Expr. Purif., 5, 1994 : 270-277). Rellos et al. describe the mutagenesis of the alcohol dehydrogenase 2 gene of Zymomonas mobilis by a PCR method without using dITP because the limitation of a nucleotide is entirely sufficient for increasing the mutation rate and thus for the required result.

The use of an in vitro recombination technique for molecular evolution is described by Stemmer (Proc. Natl. Acad. Sci. USA, Vol. 91, 1994: 10747-10751). It was possible by this recombination technique to restore the enzymatic function of the lacZ gene from two mutated and inactivated lacZ genes.

Moore et al. (Nature Biotechnology Vol. 14, 1996: 458-467) describe the combination of the PCR and recombination methods for increasing the enzymatic activity of an esterase toward a para-nitrobenzyl ester.

The disadvantages of said methods is that, in order to produce the mutations, the DNA must be treated in vitro with enzymes such as Taq polymerase and/or restriction enzymes and/or oligonucleotides and the various potential mutants require individual further treatment and testing. These methods are suitable only for mutagenesis of relatively small DNA regions.

Another route to the mutagenesis of enzymes is described by Greener et al. in Methods in Molecular Biology (Vol. 57, 1996: 375-385). Greener et al. use the specific Eschericha coli strain XL1-Red to generate Eschericha coli mutants which have increased antibiotic resistance. This increased antibiotic resistance is attributable to an increased copy number of the plasmid pBR322 which codes for a β-lactamase. This Escherichia coli strain can also be used to generate auxotrophic mutants and increase the enzyme activity.

The disadvantage of all the mutagenesis methods mentioned is that it is possible to optimize only enzymatic activities which are present. If new enzymatic reactions, ie. new substrate specificities of the enzymes, are required, for example for cleaving a new substrate, it is necessary first to search for this new enzymatic activity in an elaborate screening of natural forms. As a rule, it is then necessary to optimize the enzyme further.

It would therefore be desirable to have a method which makes it possible to generate new enzymatic activities, ie. is able to alter the substrate specificity of enzymes.

It is an object of the present invention to develop a novel widely applicable method which does not have the abovementioned disadvantages and which is able to alter the substrate specificity of enzymes rapidly and straightforwardly.

We have found that this object is achieved by a method for altering the substrate specificity of enzymes, which comprises carrying out the following steps:

a) introducing a DNA which comprises a copy of the gene coding for the enzyme into the Eschericha coli strain XL1 Red or into a functional derivative,

b) incubating the transformed Eschericha coli strain XL1 Red or its functional derivative to generate mutations in the enzyme gene,

c) transmitting the mutated DNA from the strain XL1 Red or its functional derivative to a microorganism which has no impeding enzyme activity,

d) incubating this microorganism to detect the enzyme activity on or in at least one selection medium which comprises at least one enzyme substrate which makes it possible to recognize unaltered substrate specificity of the enzyme, with or without other indicator substances,

e) selecting the microorganisms which show an alteration in the substrate specificity.

Alteration of the substrate specifity in the novel method means that the enzymes having been subjected to the method are able to convert substrates which they were previously unable to convert, because the affinity of the enzyme for the substrate was too low (=high K_{M}) and/or the catalytic activity (=k_{cat}) of the enzymes was too low. In these cases, the ratio k_{cat}/K_{M} is zero or almost zero, ie. catalysis does not occur. The alteration in the substrate specificity reduces the Km or increases the k_{cat}, or both, ie. the ratio k_{cat}/K_{M} becomes greater than zero. A catalytic reaction occurs. The enzyme converts the new substrate after the mutagenesis.

It is possible in principle for the substrate specificity of all enzymes to be altered, and preferably the substrate specificity of hydrolases is altered in the novel method. Hydrolases form the 3rd class of enzyme (=3..)in the IUB nomenclature system. Hydrolases are preferred in the novel method because, as a rule, a simple detection reaction for them exists and, in many cases, they are used in industrial syntheses. It is particularly preferred to alter the substrate specificity of hydrolases selected from the group consisting of proteases, lipases, phospholipases, esterases, phosphatases, amidases, nitrilases, ether hydrolases, peroxidases and glycosidases, very particularly preferably lipases, esterases, nitrilases or phytases.

After the substrate specificity has been altered in the method according to the invention, the enzyme reaction can take place without or with selectivity in the case of chiral starting compounds, ie. the reaction results in racemic or optically active products. Preferred alterations result in selective alterations in the substrate specificity such as regio-, chemo- or stereoselective or in regio-, chemo- and/or stereoselective reactions.

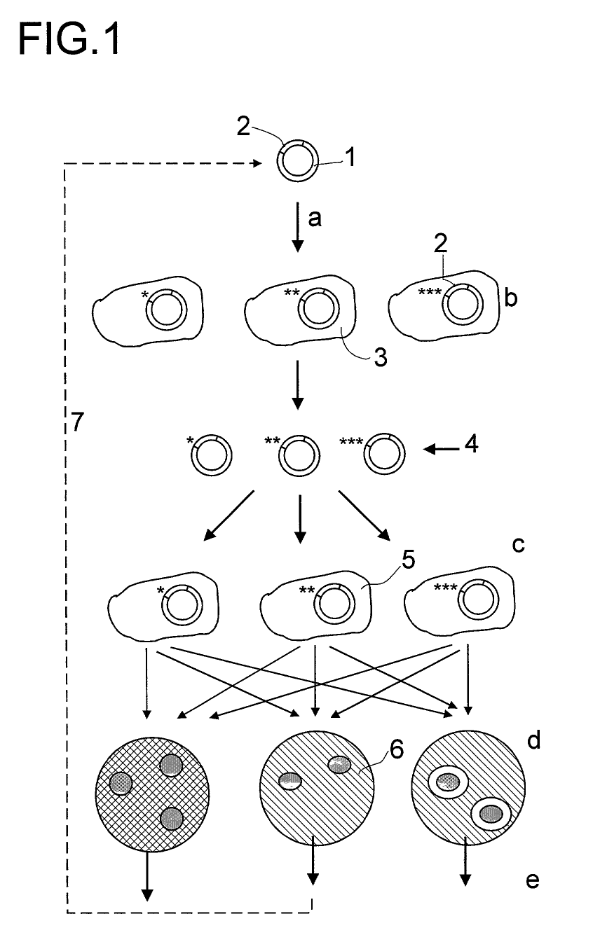

All methods known to the skilled worker for introducing DNA into microorganisms are suitable in the process according to the invention (step a, FIG. 1). The DNA can be introduced by means of phages, by transformation or by conjugation into the strain Eschericha coli XL1 Red or a functional derivative of this strain. Phages which are advantageously suitable and which may be mentioned are all temperate phages such as lambda or mu. Methods for introducing this phage DNA into the appropriate microorganism are well known to the skilled worker (see Microbiology, Third Edition, Eds. Davis, B. D., Dulbecco, R., Eisen, H. N. and Ginsberg, H. S., Harper International Edition, 1980). In the case of conjugation, the DNA can be transferred directly, ie. it is located on the conjugation-mediating plasmid such as the F factor, or it is transferred in the conjugation by means of a comobilizable vector. These methods are also known to the skilled worker. The method which may be mentioned as preferred is the introduction of the DNA by transformation (Winnacker, E. L., From Genes to Clones, VCH, 1987: 126-127, Williams et al., Ann. Rev. Gen., Vol. 14, 1980: 41-76). Numerous methods for transforming microorganisms are known to the skilled worker from the literature, and these use a wide variety of reagents such as PEG (Chung et al. Proc. Natl. Acad. Sci. USA, Vol. 86, 1989: 2172-2175), CaCl₂ (Mandel et al., J. Mol. Biol. Vol. 53, 1970: 159-162), dimethyl sulfoxide, hexamminecobalt and dithiothreitol in the presence of mono- or divalent cations (Hanahan D., J. Mol. Biol., 166, 1983: 557-580) or electroporation (see DNA Cloning 1, Ed. Glover et al., IRL Press, 1995, Hanahan et al., Technics for Transformation of E. coli, page 1-36, ISBN 0199634769).

All conventional vectors can be used for the transformation. The vectors normally used are those which are able to replicate Eschericha coli. If the microorganism to be used as selection microorganism allows the enzyme activity to be detected is to come from a different kingdom, for example fungi such as Aspergillus, Ashbya or Beauveria or yeasts such as Saccharomyces, Candida or Pichia, another family, for example Actinomycetales, or another genus such as Pseudomonas, Streptomyces, Rhodococcus or Bacillus, it is advantageous to use shuttle vectors which are able to replicate in both microorganisms, because this makes recloning of the DNA unnecessary.

Examples of vectors which may be mentioned are the following plasmids: pLG338, pACYC184, pBR322, pUC18, pUC19, pKC30, pRep4, pPLc236, pMBL24, pLG200, pUR290, pIN-III¹¹³-B1, λgt11 or pBdCI. Other vectors are well known to the skilled worker and can be found, for example, in the book Cloning Vectors (Eds. Pouwels P. H. et al. Elsevier, Amsterdam-New York-Oxford, 1985, ISBN 0 444 904018).

Particularly suitable for the methods according to the invention for producing mutations (step b, FIG. 1) is the Escherichia coli strain XL1 Red (=Epicurian coli XL-1 Red), which is marketed by Stratagene La Jolla, Calif. It carries the following genetic markers: Δ[mcrA]183, Δ[mcrCB-hsdSMR-mrr]173, endA1, supE44, thi-1, gyrA96, relA1, mutS, mutT, mutD5, lac. MutS is a mutation in the mismatch repair pathway, mutT is a mutation in the oxo-dGTP repair pathway and mutD5 is a mutation in the 3′-5′exonuclease subunit of DNA polymerase III. Competent cells of this strain can be purchased from Stratagene under order number 200129. A functional derivative of this strain preferably means Eschericha coli strains which contain the following genetic markers: relAl, mutS, mutT and mutD5. These genetic markers result in a distinctly increased mutation rate in the organisms. They should therefore not be incubated on agar plates or in a culture medium for too long, because otherwise they lose their vitality.

For detection of the altered substrate specificity (=mutations in the enzyme used) it is possible and advantageous, in the case where vectors have been used, for the DNA initially to be isolated from the E. coli strain XL1 Red or its functional derivative and be inserted into a microorganism which has no corresponding enzyme activity (step c, FIG. 1). If, for example, an esterase is introduced into these selection organisms, these microorganisms must not have any esterase activity which cleaves the ester used for selection for an alteration in the substrate specificity of the esterase. Other esterase activities in this organism do not interfere with the selection. The introduction of the DNA can take place, as described above, using phages or viruses, by conjugation or by transformation. In the case of introduction via phages or viruses or by conjugation, specific isolation of the DNA is unnecessary. The DNA can be introduced directly, by conjugation or via the phage or virus, into the microorganism used for the selection. Thus, transfer of the DNA takes place in these cases without isolation of the DNA, and in the case where vectors are used by a transformation. It is also possible for the DNA of the microorganisms which show an altered substrate specificity after selection to be introduced without isolation by conjugation or using phages or viruses or by transformation into the strain E. coli XL1 Red or a functional derivative for a further selection cycle (=FIG. 1, dotted line). It is possible in this way for the method according to the invention to be performed one or more times in sequence. The DNA is in this case transferred from the E. coli strain XL1 Red or its functional derivatives to the selection organisms and finally returns to the E. coli strains for a new selection cycle.

Selection microorganisms which are suitable in principle in the method according to the invention are all prokaryotic or eukaryotic microorganisms, although they must have no enzymatic activity which could impede the selection. This means either that the microorganisms have no enzymatic activity which is sought, ie. the substrate(s) used for selecting the altered substrate specificity are not converted by the enzymes in the selection microorganism, or that only a small enzymatic activity of this type is present in the microorganisms and still permits selection. Suitable and advantageous microorganisms for the method according to the invention are Gram-positive or Gram-negative bacteria, fungi or yeasts. Those preferably used are Gram-positive bacteria such as Bacillus, Rhodococcus, Streptomyces or Nocardia, or Gram-negative bacteria such as Salmonella, Pseudomonas or Escherichia. Escherichia coli strains are very particularly preferably used. The genus and species or the membership of a family or kingdom of the microorganisms used for the selection is of minor importance as long as it allows selection of the altered substrate specificity.

To select for altered substrate specificity, the microorganisms are incubated, for detection of the enzyme activity, on or in at least one selection medium which contains at least one enzyme substrate which makes it possible to recognize an altered substrate specificity of the enzyme (FIG. 1, step d). This selection medium may contain other indicator substances which improve recognition of the desired alteration. Possible additional indicator substances of this type are, for example, pH indicators.

FIG. 1 depicts the individual steps in the method taking the example of the use of a vector (1). Step a shows the introduction of the DNA (2) via the vector into the strain Eschericha coli XL1 Red or into a functional derivative (3) of this strain. The DNA of the enzyme is mutated in these organisms [asterisks in FIG. 1 indicate diagrammatically by way of example the mutations in the DNA (2)]. The mutated vectors (4) are then reisolated from the strain Eschericha coli XL1 Red or its functional derivatives and subsequently transformed, directly or after storage, into the selection organisms (5) (step c). These organisms are finally plated out on at least one selection medium (6) and thus the altered enzyme substrate specificity is identified by, for example, a growth assay and/or a visual assay (step d). Positive clones which show an altered substrate specificity are finally selected, and the mutated gene coding for the altered enzyme can be isolated (step e). The method can be repeated several times using the mutated gene FIG. 1, dotted line (7)].

The microorganisms used in the method according to the invention, ie. the Eschericha coli XL1 Red strain and its functional derivatives, and the selection organisms used are cultured in a medium which allows these organisms to grow. This medium can be a synthetic or a natural medium. Media known to the skilled worker and depending on the organism are used. For growth of the microorganisms, the media used contain a source of carbon, a source of nitrogen, and inorganic salts with or without small amounts of vitamins and trace elements.

Examples of advantageous sources of carbon are sugars such as mono-, di- or polysaccharides such as glucose, fructose, mannose, xylose, galactose, ribose, sorbose, ribulose, lactose, maltose, sucrose, raffinose, starch or cellulose, complex sources of sugars such as molasses, sugar phosphates such as fructose 1,6-bisphosphate, sugar alcohols such as mannitol, polyols such as glycerol, alcohols such as methanol or ethanol, carboxylic acids such as citric acid, lactic acid or acetic acid, fats such as soybean oil or rapeseed oil, amino acids such as glutamic acid or aspartic acid or amino sugars, which can also be used at the same time as source of nitrogen.

Advantageous sources of nitrogen are organic or inorganic nitrogen compounds or materials which contain these compounds. Examples are ammonium salts such as NH₄Cl or (NH₄)₂SO₄, nitrates, urea, or complex sources of nitrogen such as corn steep liquor, brewer's yeast autolysate, soybean meal, wheat gluten, yeast extract, meat extract, casein hydrolysate, yeast or potato protein, which can often also serve at the same time as source of carbon.

Examples of inorganic salts are the salts of calcium, magnesium, sodium, manganese, potassium, zinc, copper and iron. The anion of these salts which should be particularly mentioned is the chloride, sulfate and phosphate ion.

Where appropriate, further growth factors are added to the nutrient medium, such as vitamins or growth promoters such as riboflavin, thiamine, folic acid, nicotinic acid, pantothenate or pyridoxine, amino acids such as alanine, cysteine, asparagine, aspartic acid, glutamine, serine, methionine or lysine, carboxylic acids such as citric acid, formic acid, pimelic acid or lactic acid, or substances such as dithiothreitol.

To stabilize the DNA present in the vectors or phages in the cells it is possible, where appropriate, to add antibiotics to the medium.

The mixing ratio of said nutrients depends on the mode of incubation (=fermentation) and is established in the individual case. The medium components may all be present at the start of the fermentation after they have been sterilized, if necessary, separately or together, or else can be added as required during the incubation.

Plate media are preferred to liquid media because they make it easier to detect the required alteration in the substrate specifity. It is important that none of the medium components used could interfere with the detection of the altered enzyme activity.

The culturing conditions are established so that the organisms (=Eschericha coli strain XL1 Red and selection organisms) grow optimally and that the best possible yields are obtained. Culturing is preferably carried out at from 15° C. to 40° C., particularly advantageously from 25° C. to 37° C. The pH is preferably kept in the range from 3 to 9, particularly advantageously from 5 to 8. In general, the incubation time of from 1 to 240 hours, preferably from 5 to 170 hours, particularly preferably from 10 to 120 hours, is sufficient, but longer incubation times may also be necessary in a few cases for the mutagenesis or detection.

The altered substrate specificity can, after identification of the corresponding clones, advantageously be checked again in an in vitro assay.

### EXAMPLES

Retrosynthetic analysis showed that the 3-hydroxy esters [see formula II, (1) and (3)] are favorable starting material for synthesizing the macrolide antibiotic epothilon A (see formula I).

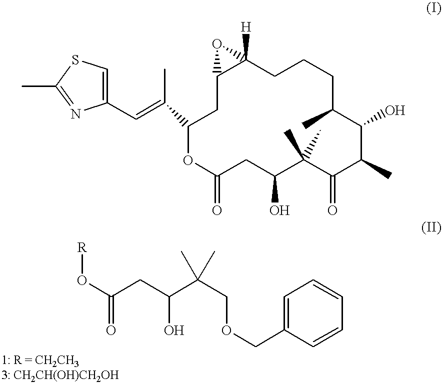

The optically active preparation of the compounds followed an enzymatic route using esterases or lipases. Surprisingly, none of the 18 lipases and two esterases used showed enzymatic activity toward the 3-hydroxy esters (see Table I). No enzymatic activity was detectable either in the hydrolysis direction, which was carried out in a phosphate buffer, or in the synthesis direction, which was carried out with vinyl acetate in toluene.

TABLE I
Tested lipases and esterases - hydrolysis of the
ethyl ester (II, 1)

| Enzyme from | Manufacturer |
|---|---|
| Pseudomonas cepacia (= PS) | Amano, Nagoya, Japan |
| Pseudomonas cepacia (= AH) | Amano, Nagoya, Japan |
| Acylase (ACS) | Amano, Nagoya, Japan |
| Rhizopus delamar (D) | Amano, Nagoya, Japan |
| Rhizopus javanicus (F-AP 15) | Amano, Nagoya, Japan |
| Candida rugosa (AY) | Amano, Nagoya, Japan |
| Mucor javanicus (M) | Amano, Nagoya, Japan |
| Penicillium roquefortii (R) | Amano, Nagoya, Japan |
| Penicillium cyclopium (G50) | Amano, Nagoya, Japan |
| Chromobacterium viscosum (crude) | Toyo Jozo, Tokyo, Japan |
| Chromobacterium viscosum (pure) | Toyo Jozo, Tokyo, Japan |
| Rhizomucor miehei | Biocatalysts Ltd. Ponty- |
| | pridd, UK |
| Humicola lanuginosa | Biocatalysts Ltd. Ponty- |
| | pridd, UK |
| Rhizomucor miehei (Lipozyme, | Novo, Bagsvaerd, Denmark |
| immob.) | |
| Candida antarctica B (SP435, | Novo, Bagsvaerd, Denmark |
| immob.) | |
| Candida antarctica B (SP525, free) | Novo, Bagsvaerd, Denmark |
| Candida antarctica A (SP526, free) | Novo, Bagsvaerd, Denmark |
| Candida antarctica A, B (SP382, | Novo, Bagsvaerd, Denmark |
| free) | |
| Pig liver esterase | Fluka, Buchs, Switzerland |
| Esterase from Thermoanaerobium | Fluka, Buchs, Switzerland |
| brockii | |

In order to make conversion of these compounds (II) possible, therefore, we followed a mutagenesis strategy starting from an esterase from Pseudomonas fluorescens which had been cloned into E. coli. The cloning and sequencing of this esterase was described for the first time by Choi et al. (Agric. Biol. Chem. Vol. 54, No. 8, 1990: 2039-2045). Pelletier and Altenbuchner (Microbiology, Vol. 141, 1995: 459-468) have resequenced this esterase and found some errors in the original sequence of Choi et al.

The esterase was mutagenized using the strain Eschericha coli XL-1 Red (=Epicurian coli XL-1 Red).

1. Preparation of the esterase and of the mutated esterases

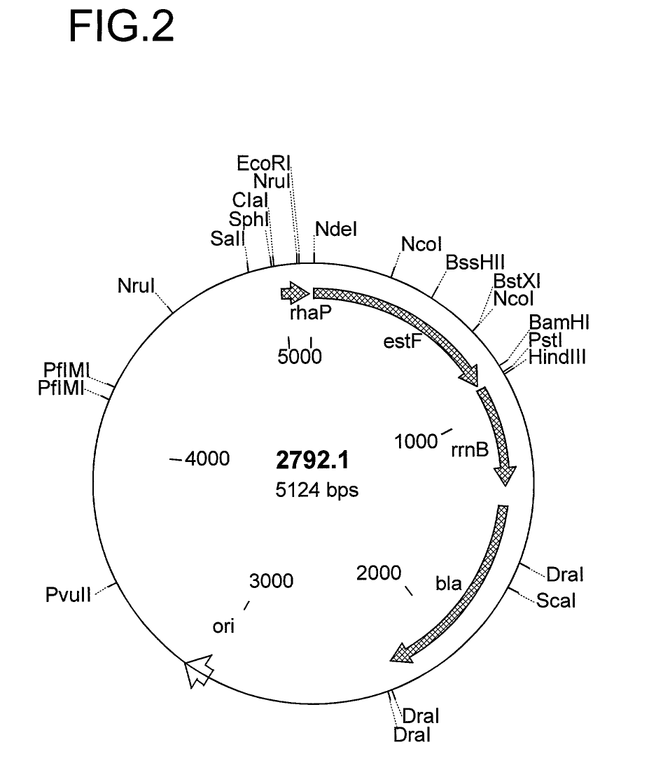

The strain Eschericha coli JM109 or DH5α which harbors the rhamnose-inducible plasmid 2792.1 (see FIG. 2) was grown in Luria-Bertani(=LB) medium supplemented with 100 μg/ml ampicillin at 37° C. up to the early expontential phase (OD₆₀₀ 0.5-0.6, about 3 hours). The esterase gene estF is located on the plasmid 2792.1. Gene expression was induced at the time by adding rhamnose [final concentration 0.2% (w/v)] to the culture. The cells were then incubated at 37° C. for a further 3.5 h, harvested by centrifugation (5000 rpm, 5 min, 4° C.) and washed twice with potassium phosphate buffer (50 mM, pH 7.5, 4° C.). The cells were then resuspended in the same buffer and disrupted with ultrasound. The cell detritus was removed by centrifugation (5000 rpm, 15 min, 4° C). The protein concentration in the supernatant was measured using the bicinchoninic acid protein determination kit supplied by Pierce, Rockford, Ill., USA (order number 23223, U.S. Pat. No. 4,839,295). The specific activity of the PFE was determined by photometry or in a pH-stat (see 2. esterase activity). The PFE was used directly in the hydrolysis experiments. The PFE was not purified further for the hydrolysis experiments because the esterase produced by the strains was already of high purity. Simple purification by zinc affinity chromatography resulted in a homogeneous esterase solution. This increased the specific activity only slightly.

2. Determination of esterase activity

The esterase activity was determined in a pH-stat assay with 5% (w/v) ethyl acetate in an emulsion which, besides distilled water, contains 2 % (w/v) gum arabic, at 37° C. and pH 7.5. A known amount of esterase was added to 20 ml of this emulsion. The liberated acetic acid was titrated against 0.1 N NaOH automatically in a pH-stat supplied by Metrohm (Herisau, Switzerland) so that the pH remained constant. The esterase activity has been reported in units, where one unit (=U) is defined as the amount of enzyme which produces 1 μmol of acetic acid per minute under the assay conditions. The measured number of units was corrected by the figure for the autolysis of ethyl acetate under these conditions, which is 0.25 μmol per minute at 37° C.

3. Biotransformation

The 3-hydroxy esters were hydrolyzed in round-bottom flasks with stirring at 700 rpm. 0.5 mmol of the ester 1 or 3 was added to a milliliter of toluene at 37° C. The reaction was started by adding 3 ml of culture supernatant and stopped by extracting with ether. The organic phase was dried over MgSO₄. The substrates and the product were separated using a silica gel column (petroleum ether: ether, 2:1).

To analyze the reaction products, 100 μl of the reaction mixture were centrifuged and the supernatant was analyzed using a thin layer (silica gel plates, visualized with UV or spraying with cerium reagent) or GC (Hewlett Packard, model HP5890 series II) using a chiral column [=heptakis(6-O-thexyldimethylsilyl-2,3-di-O-methyl)-β-cyclodextrin, 25×0.25 mm, 15 m, Prof. W. König, Institute of Organic Chemistry, Hamburg University, Germany]. The GC analysis was carried out at 125° C. (isothermal) with helium as carrier gas (80 kPa), with a flame ionization detector and with a split ratio of 1:100.

4. Cultivation of the microorganisms

The microorganisms used (mutagenesis strain: Eschericha coli XL-1 Red, selection strain: Eschericha coli DH5α) were cultured in LB medium, LB/Amp medium [LB supplemented with ampicillin (1% w/v)], LB/TB/Amp medium (LB supplemented with tributyrin (1% v/v) and ampicillin (1% w/v) and 1% agar] and MM/Amp/Ind/Rha [minimal medium supplemented with ampicillin (1% w/v) and, as indicator substances, crystal violet (1 mg/l) and neutral red (30 mg/L) and for induction rhamnose (0.2% w/v) plus 1% agar]. The composition of LB medium and of minimal medium (M9) are to be found in Maniatis et al., Molecular Cloning: A Laboratory Manual (Sec. Edition, Vol. I, II, III, Cold Spring Harbor Laboratory Press, 1989, ISBN 0-87969-309-6) or Greener et al. (Methods in Molecular Biology, Vol. 57, 1996: 375-385).

5. Mutagenesis

The mutagenesis was carried out as depicted in FIG. 1. The plasmid PFE-WT was isolated from an overnight culture in an LB/Amp medium using a kit supplied by Quiagen (Hilden, Germany) and then, for the mutagenesis, transformed into competent cells of the strain Epicurian coli XL-1 Red and cultured in 50 ml of LB/Amp medium supplemented with 20 mM MgCl₂ and 20 mM glucose at 37° C. overnight. 500 μl of this culture were incubated in fresh LB/Amp medium (50 ml) and subjected to another mutation cycle. 2 ml of cells were taken from the remaining cultures and were centrifuged (3000 rpm, 10 min, 4° C.) and the plasmid was isolated from these cells (see Maniatis et al., Molecular Cloning: A Laboratory Manual, Sec. Edition, Vol. I, II, III, Cold Spring Harbor Laboratory Press, 1989, ISBN 0-87969-309-6). Up to seven mutation cycles were carried out successively. Positive mutants identified in the first round (eg. PFE-U3) were subjected to a second mutagenesis round (up to seven cycles).

6. Transformation of the microorganisms

The microorganisms E. coli XL1 Red or E. coli DH5α were transformed by the method described by Chung et al. (Proc. Natl. Acad. Sci. USA, Vol. 86, 1989: 2172-2175). After the transformants had been cultured in LB/Amp medium at 37° C. for about 1 hour, aliquots (50-100 μl) of the culture were plated out on LB/Amp agar plates and incubated at 37° C. overnight. These plates were used as masterplates for the subsequent replica plating as described by Lederberg in the screening experiments (Manual of Methods for General Bacteriology, American Society For Microbiology, Washington, DC 20006, ISBN 0-914826-29-8). In addition, 50 μl of culture were plated out on LB/TB/Amp plates which contained 0.2% rhamnose to identify colonies producing an active esterase.

7. Screening system

To identify the mutated genes, the colonies underwent replica plating from the masterplates onto the following selection plates (Manual of Methods for General Bacteriology, American Society For Microbiology, Washington, DC 20006, ISBN 0-914826-29-8): a) MM/Amp/Ind/Rha with 0.1% (w/v) substrate (compound 1), b) MM/Amp/Ind/Rha with 0.1% (w/v) substrate (compound 3). The plates were incubated at 37° C. Positive clones were identified on the basis of the red color around and in the colonies—lowering of the pH—on plate types (a) and (b) and fast growth on the (b) plates. A red color is to be seen with all colonies after 2 days on LB/Amp/Ind plates. This is presumably attributable to a side reaction. Thus only the abovementioned minimal medium plates are suitable for the assays. Comparison with the masterplates was carried out to select only the colonies which gave a positive result in both methods, ie. both produced an intact esterase and showed a red color. From about 750 colonies in the first mutagenesis round, two positive clones (=PFE-U1 and PFE-U3) were identified after incubation at 37° C. for 2 to 6 days. In the case of PFE-U1 only a weak red color was produced, whereas in the case of PFE-U3 a deep red color was produced and compound 3 showed good growth. The vectors (=plasmids) of these clones were isolated and transformed again into the strain E. coli DH5α. These organisms were used to produce mutated esterases after rhamnose induction in 250 ml of LB/Amp medium. The esterase (see above) was isolated and used for preparative conversion of compound 1. The unmutated wild-type esterase was used as control.

8. Nucleotide sequence analysis

The nucleotide sequence of the esterase genes was determined using a fluorescence dideoxy DNA sequencing method. The DNA sequencing was carried out using the Taq Dye Deoxy™Cycle sequencing kit (Applied Biosystems, Weiterstadt, Germany) in accordance with the manufacturer's instructions and using primers derived from the nucleic acid sequence (Interactiva, U1m, Germany). Sequencing of the PFE-U1 gene revealed no mutation in the structural gene, whereas the sequence of PFE-U3 showed two point mutations. This resulted in an amino acid exchange in position 209 (A by D) and in position 181 (L by V). Modeling analyses showed that the amino acid exchanges are remote from the active center.

9. Chemical synthesis of compounds 1 and 3

a) Ethyl 5-(benzyloxy)-3-hydroxy-4,4-dimethylpentanoate (=compound 1)

An ethyl acetate solution (8.8 g, 100 mmol in 10 ml of THF) was added dropwise to a solution of 170 ml of lithium diisopropylamide (110 mmol, prepared from 11.1 g of diisopropylamine and 68.8 ml of butyllithium, 1.6 N in hexane) in THF/hexane at −60° C. After 30 minutes, 30 ml of 3-benzyloxy-2,2-dimethylpropanal (19.2 g, 100 mmol) in THF were rapidly added, and mixture was stirred for 5 minutes before an aqueous potassium bisulfate solution was added to neutralize. The reaction mixture was extracted with diethyl ether, washed several times with water and dried over magnesium sulfate, and the organic solvent was removed under reduced pressure. 26.1 g (93%) of the compound were isolated a pale yellow oil. The compound was purified on a silica gel column (ether petroleum ether, 1:3 to 1:1).

¹H-NMR(CDCl₃): δ=0.90 (s,CH₃), 0.93 (s,CH₃), 1.25 (t,J=7 Hz, CH₃), 2.37 (dd, J=16 Hz, J=10 Hz, 1H), 2.49 (dd, J=16 Hz, J=4 Hz, 1H), 3.28 (d, J=9 Hz, 1 H), 3.36 (d,J=9 Hz, 1H), 3.55 (d, J=4 Hz, OH), 4.02 (dt, J=10 Hz, J=4 Hz, 1H), 4.16 (q,J=7 Hz, CH₂), 4.49 (s,BnCH₂), 7.30 (m, 5 aryl-H)ppm. ¹³C-NMR(CDCl₃): δ=14.16, 19.78, 22.10, 37.23, 38.15, 60.50, 73.42, 74.00, 78.44, 127.57, 128.34, 138.05, 173.15 ppm. IR (cap. film): ν=3500 (broad), 3064, 3028, 2976, 2936, 2904, 2872, 1732, 1476, 1452, 1368, 1308, 1256, 1184, 1156, 1096 cm⁻¹. MS(RT): m/e=280 (0.5 M+), 234(1), 205(1), 190(3), 174(1), 159(2), 156(1), 141(4), 117(9), 111(3), 109(6), 108(13), 107(16), 91(100), 79(10), 71(12).

b) 2,3-Dihydroxypropyl 5-(benzyloxy)-3-hydroxy-4,4-dimethylpentanoate (=compound 3)

The compound 2,2-dimethyl-1,3-dioxolan-4-yl)methyl 5-(benzyloxy)-3-hydroxy-4,4-dimethylpentanoate (=compound 2) was synthesized as described under a using mmol of lithium diisopropylamide, 30 mmol of 1-acetyl-2,3-isopropylideneglycerol and 30 mmol of 3-benzyloxy-2,2-dimethylpropanal. Compound 3 was synthesized starting from 3.66 g (10 mmol) of compound 2 in 30 ml of methoxyethanol. The solution of compound 2 was heated with 5.0 g of boric acid at 110° C. for 20 minutes. After removal of the solvent, the residue was mixed with small amounts of water and extracted with diethyl ether. The extract was dried over magnesium sulfate, the solvent was removed under reduced pressure and compound 3 was purified by silica gel chromatography (ether and ether:dioxane, 9:1). 2.3 g (70%) of compound 3 were isolated as colorless oil in a 1:1 diastereomer mixture.

¹H-NMR(CD₃OD): δ=0.90 (s,CH₃), 0.92 (s,CH₃), 2.40 (m, 1H), 2.59 (m, 1H), 3.24 (d, J=9 Hz, 1 H), 3.33 (d,J=9 Hz, 1H), 3.56 (m, CH₂), 3.84 (quint, J=5 Hz, 1H), 3.99-4.22 (sev. m, 3H), 4.45 (d,J=12 Hz, 1H)), 4.51 (d,J=12 Hz, 1H) 7.31 (m, 5 aryl-H)ppm. ¹³C-NMR(CD₃OD): δ=21.00, 21.63, 38.52, 39.82, 64.00, 64.02, 66.64, 66.68, 71.08, 71.10, 74.07, 74.35, 78.21, 128.56, 128.67, 129.35, 139.94, 174.41 ppm. IR (CHCl₃): ν=3600 (broad), 3436 (broad), 3088, 3064, 3000, 2964, 2876, 1732, 1600, 1452, 1412, 1384, 1364, 1308, 1284, 1256, 1228, 1180, 1156, 1088, 1052 cm⁻¹. MS (140 ° C.) : m/e=326(0.5, M⁺), 308(1), 235(4), 202(5), 188(3), 174(3), 163(9), 145(19), 108(19), 107(11), 91(100), 71(12).

10. Enzymatic hydrolysis with PFE-U3

Enzymatic hydrolysis of racemic compound 1 with the mutated esterase PFE-U3 resulted in the corresponding acid [α]D²⁰=−11.01° (c=0.965, CHCl₃), oil, 11.6% y and the remaining starting material (24.9%ee), [α]D²⁰=+0.970 (c=3.071, CHCl₃), oil, 61.4% y. Hydrolysis thus took place with a stereoselective discrimination of 1. The wild-type hydrolase as control is unable, just like the mutant PFE-U1, to convert compound 1. Mutants of the second generation starting from PFE-U3 showed no improvement in the stereoselectivity in the hydrolysis reaction. Mutants of the second generation are PFE-3-311 to PFE-3-713 (see Table II). Table II shows the specific activity of the various esterases in units with ethyl acetate as substrate (Table II, U/mg protein). The optical activity of the various esterases is based on compound 1 (see Table II).

TABLE II
Substrate specificity of various esterases

| | | Enantiomeric excess |
|---|---|---|
| | Activity (U/mg) for | (% ee based on |
| Esterase | ethyl acetate | compound 1) |
| PFE-WT | 6.0 | 0 |
| PFE-U1 | 4.9 | 0 |
| PFE-U3 | 2.3 | 24.9 |
| PFE-U3-311 | 0.9 | 3.3 |
| PFE-U3-411 | 1.3 | 7.0 |
| PFE-U3-412 | 2.1 | 3.7 |
| PFE-U3-413 | 2.5 | 3.5 |
| PFE-U3-511 | 0.9 | 3.3 |
| PFE-U3-512 | 4.4 | 3.1 |
| PFE-U3-513 | 1.5 | 3.3 |
| PFE-U3-514 | 4.1 | 4.3 |
| PFE-U3-515 | 1.1 | 3.7 |
| PFE-U3-711 | 2.7 | 3.4 |
| PFE-U3-712 | 2.3 | 3.2 |
| PFE-U3-713 | 1.3 | 3.2 |

We claim:

1. A method for altering the substrate specificity of enzymes, which comprises carrying out the following steps:
a) introducing a DNA which comprises a copy of the gene coding for the enzyme into the Eschericha coli strain XL1 Red or into a functional derivative,
b) incubating the transformed Eschericha coli strain XL1 Red or its functional derivative to generate mutations in the enzyme gene,
c) transmitting the mutated DNA from the strain XL1 Red or its functional derivative to a microorganism which has no impeding enzyme activity,
d) incubating this microorganism to detect the enzyme activity on or in at least one selection medium which comprises at least one enzyme substrate which makes it possible to recognize unaltered substrate specificity of the enzyme, with or without other indicator substances,
e) selecting the microorganisms which show an alteration in the substrate specificity.

2. A method as claimed in claim 1, wherein steps (a) to (e) are performed several times in sequence by returning the DNA from the microorganisms selected in step (e) to the strain Eschericha coli XL1 -Red or its functional derivative.

3. A method as claimed in claim 1 or 2, wherein prokaryotic or eukaryotic microorganisms are used as microorganisms.

4. A method as claimed in any of claims 1 to 3, wherein Gram-positive or Gram-negative bacteria, fungi or yeasts are used as microorganisms.

5. A method as claimed in any of claims 1 to 4, wherein a hydrolase is used as enzyme.

6. A method as claimed in any of claims 1 to 5, Wherein a hydrolase selected from the group consisting of proteases, lipases, phospholipases, esterases, phosphatases, amidases, nitrilases, ether hydrolases, peroxidases and glycosidases is used as enzyme.

7. A method as claimed in any of claims 1 to 6, wherein a lipase, esterase, nitrilase or phytase is used.

8. A method as claimed in any of claims 1 to 7, wherein the alteration in the substrate specificity results in a selective enzymatic activity.

9. A method as claimed in claim 8, wherein the alteration in the substrate specificity results in a regio-, chemo- or stereoselective or regio-, chemo- and/or stereoselective enzymatic activity.
